(19) United States
(12) Patent Application Publication
(10) Pub. No.: US 2002/0137785 A1
(43) Pub. Date: Sep. 26, 2002

(54) Inflammatory mechanism modulator composition and methods with anti-asthmatic properties

(76) Inventors: George Kindness, Middletown, OH (US); Brooke Schumm III, Ellicott City, MD (US); F. Timothy Guilford, Palo Alto, CA (US)

Correspondence Address: BROOKE SCHUMM, III, DANEKER, MCINTIRE, SCHUMM, PRINCE, GOLDSTEIN, ET A, 210 N CHARLES ST, SUITE 800, BALTIMORE, MD, 21201, US

(21) Appl. No.: 10/106,625

(22) Filed: Mar. 26, 2002

Related U.S. Application Data
(60) Provisional application No. 60/278,725, filed on Mar. 26, 2001.

(51) Int. Cl. A61K 31/496; A61K 31/47; A61K 31/433; A61K 31/417; A61K 31/341; A61K 31/198

(52) U.S. Cl. 514/400; 514/362; 514/255.04; 514/311; 514/562

(57) ABSTRACT

The invention proposes the use of a leukotriene antagonist, particularly a montelukast sodium compound such as SINGULAIR, in combination with cystine to combat inflammatory disease and hopefully reduce the necessary use of SINGULAIR. Combination with other anti-inflammatory agents and anti-asthmatic agents is proposed. Selenium to assure glutathione pathway benefit is suggested. The addition of a selective COX-2 inhibitor is suggested.

### CONTINUATION DATA

This application is a continuation in part of Provisional Application of the same name filed on Mar. 26, 2001 having Prov. Appl. No. 60/278,725, and of the same name filed Mar. 26, 2002.

### SUMMARY OF INVENTION

The inventors propose as a preferred mode a combination of a leukotriene antagonist, including a leukotriene receptor antagonist, in particular montelukast sodium, sold under the name SINGULAIR (a registered trademark of Merck & Co.), available nationally in the U.S., and cystine, or cystine like compound as herein defined. The package insert for SINGULAIR is adopted by reference and attached hereto. SINGULAIR is known as montelukast sodium and is disclosed in U.S. Pat. No. 5,565,473 and referred to as Formula I. More generally, the invention proposes to use cystine and any leukotriene inhibitor.

Other than what are more generally known as leukotriene antagonists (including SINGULAIR) are set forth in Arison, U.S. Pat. No. 5,952,347, Sep. 14, 1999. Arison, U.S. Pat. No. 5, 952,347 describes other leukotriene antagonists as quinoline diacid compounds having activity as leukotriene antagonists. Other inhibitors of the biosynthesis of the leukotrienes such as are disclosed in EP 138,481 (Apr. 24, 1985), EP 115,394 (Aug. 8, 1984), EP 136,893 (Apr. 10, 1985), and EP 140,709 (May 8, 1985), which are hereby incorporated herein by reference and may be used in combination in this invention with cystine, or as indicated in this invention, with magnesium sulfate or chloride. Also useful for this invention with cystine are leukotriene antagonists such as those disclosed in EP 106,565 (Apr. 25, 1984) and EP 104,885 (Apr. 4, 1984) which are hereby incorporated herein by reference and others known in the art such as those disclosed in EP Application Nos. 56,172 (Jul. 21, 1982) and 61,800 (Jun. 10, 1982); and in U.K. Patent Specification No. 2,058,785 (Apr. 15, 1981), which are hereby incorporated herein by reference.

The invention is useful as anti-asthmatic, anti-allergic, anti-inflammatory, and cytoprotective agents. This invention is useful for the treatment of diseases set forth in Arison, U.S. Pat. No. 5,952,347, Sep. 14, 1999, namely angina, cerebral spasm, glomerular nephritis, hepatitis, endotoxemia, uveitis, and allograft rejection. In addition to those diseases referenced in Arison, the invention is applicable to angina and vascular spasm dysfunction and certain arrythimia problems, as well as alleviate 5-hydroxy tryptamine (5-HT) and serotonin mediated mechanisms by at least modifying inflammatory symptoms, through regulation of cytokine activated responses, including migraine and migraine-like conditions, and to ameliorate neurodegenerative diseases aggravated by inflammatory condition and carotidynia (generally all of these diseases, maladies and actions as agent(s), including those referenced later in this description, are referred to in this invention as “applications” or “diseases”). The novelty is that in addition to the properties of SINGULAIR, the anti-inflammatory properties of cystine in combination, and in particular the shift in signaling mechanisms, particularly the notable response from Th2 to Th1, are particularly efficacious in ameliorating the diseases referenced.

For the preferred mode, the first objective is to inhibit the initiation of asthmatic response.

The second objective is to minimize inflammatory response and strengthen the body's immune system, and minimize pro-inflammatory biochemical stimuli while maximizing anti-inflammatory biochemical physiologic stimuli.

To enhance the anti-inflammatory effect, a selective COX-2 inhibitor may be utilized as well.

For emergency situations in particular, the administration of magnesium sulfate or magnesium chloride, which functions as a smooth muscle relaxant, is proposed. The combination of intravenous SINGULAIR, cystine, and magnesium sulfate or magnesium chloride can promptly ameliorate a severe asthmatic episode.

The substances may be combined by pharmaceutically acceptable methods. Cystine is the form of cysteine normally circulating in the bloodstream, and is stably absorbed upon oral administration. A reasonably skilled practitioner in the art of pharmacology can combine the compounds in a pharmacologically acceptable carrier.

### PHARMACOLOGICAL COMPOUNDS IN THIS INVENTION

The meaning of leukotriene receptor antagonist has the meaning of SINGULAIR and the reference in the package insert to the more general class of leukotriene antagonists which are substances which inhibit the cysteinyl leukotriene CysLT₁ receptor. U.S. Pat. No. 5,952,347, Sep. 14, 1999, Arison, is referred to for details and reference on formulation. It is sold by Merck & Co. of Whitehouse Station, N.J., throughout the United States by prescription in pharmacies.

Cystine will be the collective reference for glutathione pathway enhancing compounds in this description. Those cystine compounds include N-acetyl-cysteine which is normally referred to as NAC, but in this invention, the term cystine, and the term glutathione precursor, also includes the following:

Cystine is (3,3′-dithiobis [2-aminopropanoic acid]). Cystine is readily reduced to cysteine. Cystine is present in most mammalian hair and keratin.

Cysteine is 2-amino-3-mercapto propanoic acid. It is readily converted by oxioreduction to cystine. It is a constituent of glutathione and abundantly present in the metallothioneines.

Cystine in the body-useful form as L-cystine is available from Spectrum Chemical Mfg. Corp. 14422 S. San Pedro St., Gardena, Calif. 90248.

Cystine, cysteine, and N-Acetyl cysteine and pharmaceutically acceptable salts, including the pharmaceutically active forms described in Kozhemyakin et al, published by WIPO as WO 00/031120, PCT/RU99/00453, filed internationally on Nov. 19, 1999, “Hexapeptide with the Stabilized Disulfide Bond and Derivatives Thereof Regulating Metabolism, Proliferation, Differentiation and Apoptosis,” will all collectively be referred to as cystine in this invention. Other glutathione pathway enhancing compounds understandable to one of ordinary skill in the art which are encompassed in the term cystine are stable forms of compounds that enhance the glutathione pathway, the substituents of which are suggested in Kozhemyakin et al, Hexapeptide with the Stabilized Disulfide Bond and Derivatives thereof Regulating Metabolism, Proliferation, Differentiation and Apoptosis published as WO 00/31120, Jun. 2, 2000. Included in the term cystine is also any therapeutically beneficial sulfur-donating compound, such as lipoic acid and including ebselen and s-acetyl-glutathione, which interacts with the glutathione pathway and the monoethyl ester of glutathione (in which the glycine carboxyl group is esterified) (Puri R N, Meister A. “Transport of glutathione, as gamma-glutamylcysteinylglycyl ester, into liver and kidney”, Proc Natl Acad Sci USA, Vol. 80(17):5258-60, September 1983). The invention contemplates in the term cystine undenatured whey protein products designed to have enhanced cystine concentration as well as protein products that contain cysteine and cystine. They can be in the form of food products.

The addition of cystine, cysteine, N-acetyl cysteine, or the pharmaceutically acceptable salt of those substances yields another effect in this invention not facially evident from the independent properties of the basic components of the invention (hereafter each substance or a pharmaceutically acceptable salt is referred to as a “cystine”). Administration of a cystine family member, preferably cystine, which has the best and most rapid upload into the glutathione pathway and better storage capability by the body, or N-acetyl cysteine, enhances the immune system competency of the patient.

In individuals on prophylactic therapy the cystine can be continued for extended periods with oral ingestion of NAC or a cystine source such as undenatured whey protein such as Immunocal (a Registered Trademark of a product manufactured by Immunotec, Montreal Canada). Immunocal® undenatured whey protein has the added advantage of providing the cysteine in the disulfide form, called cystine. 80% of the circulating cysteine in the body is in the form of cystine. Cystine is readily absorbed into cells and has been demonstrated to be preferred by certain cells such as astrocytes (Kranich O et al, “Utilization of cysteine and cysteine precursors for the synthesis of glutathione in astroglial cultures: preference for cystine,” Glia. Vol. 22(1):11-8 January 1998.).

The present invention discloses the use of specific antioxidants delivered as oral therapies, systemic infusions or via inhalation therapies. The effectiveness of the therapy can be enhanced using blood sample monitoring of glutathione levels. Glutathione can be measured in the serum, white blood cell or red blood cell. There is no risk to elevating the level of the precursors of glutathione such as NAC as gamma-glutamylcysteine the direct precursor of glutathione is regulated by the enzyme gamma-glutamyl synthase. The feedback regulator of glutathione production in the cell is the level of glutathione. If glutathione is low and glutathione precursors are available, the cell mechanisms will form glutathione. If adequate glutathione is present, additional glutathione is not produced. Cysteine or cystine has been shown to be the rate-limiting factor in the production of glutathione (Meister A, “New aspects of glutathione biochemistry and transport-selective alteration of glutathione metabolism,” Nutrit Rev 1984; 42:397-410). Monitoring the level of glutathione will maximize the dosing schedule for the glutathione precursor being used.

The use of laboratory assessment of the glutathione levels and antioxidant capacity is useful in order to maximize the therapy.

The invention claims the use of selective COX-2 inhibitor, including rofecoxib or celecoxib, but the principles stated are generally applicable to all selective COX-2 inhibitors.

The meaning and definition of Cyclooxygenase-2 inhibitor (“COX-2 inhibitor” or “selective COX-2 inhibitor”) in this invention shall include the following in this paragraph: all of the compounds and substances beginning on page 8 of Winokur WO99/20110 as members of three distinct structural classes of selective COX-2 inhibitor compounds, and the compounds and substances which are selective COX-2 inhibitors in Nichtberger, U.S. Pat. No. 6,136,804, Oct. 24, 2000, entitled “Combination therapy for treating, preventing, or reducing the risks associated with acute coronary ischemic syndrome and related conditions”, and the compounds and substances which are selective COX-2 inhibitors in Isakson et al, PCT application WO/09641645 published Dec. 27, 1996, filed as PCT/US/9509905 on Jun. 12, 1995, entitled “Combination of a Cyclooxygenase-2 Inhibitor and a Leukotriene B4 Receptor Antagonist for the Treatment of Inflammations,” and in Waldstreicher, WO 01/45698, filed Dec. 18, 2000, published Jun. 28, 2001 entitled “Combination Therapy for Treating Neurodegenerative Disease.” Because the common names of some of the selective COX-2 inhibitor compounds are not given in Winokur, PCT/WO99/20110, Nichtberger, U.S. Pat. No. 6,136,804, Isakson, PCT WO/09641645, and Waldstreicher, WO01/45698, the meaning of COX-2 inhibitor in this invention includes compounds that are selective COX-2 inhibitors, such as NS398 and DFU (see, YERGEY, JAMES A., et al., “In Vitro Metabolism of the COX-2 Inhibitor DFU, Including a Novel Glutathione Adduct Rearomatization,” Drug Metabolism and Disposition 29(5): 638-644 (The American Society for Pharmacology and Experimental Therapeutics 2001), also known as 5,5-dimethyl-3-(3-fluorophenyl)-4-(4-methylsulphonyl)phenyl-2(5H)-furanone. The meaning of COX-2 inhibitor in this invention includes compounds that are selective COX-2 inhibitors referenced in Fosslein, “Biochemistry of Cyclooxygenase (COX)-2 Inhibitors and Molecular Pathology of COX-2 in Neoplasia,” Crit. Rev. in Clin. Labor. Sci. 37(5):431-502 (CRC Press LLC 2000). The meaning of COX-2 inhibitor in this invention also includes rofecoxib, and celecoxib, marketed as VIOXX and CELEBREX by Merck and Searle/Pfizer respectively. Rofecoxib is discussed in Winokur, WO99/20110 as compound 3, on p.9. Celecoxib is discussed as SC-58635 in the same reference, and in T. Penning, Synthesis and biological evaluation of the 1,5-diarylpyrazole class of cyclooxygenase-2 inhibitors: identification of 4-[5-(4-methylphenyl)-3-(trifluoromethyl)-1H-pyrozol-1-yl]benzenesulfonamide (SC-58635, celecoxib)”, J. Med. Chem. Apr. 25, 1997: 40(9): 1347-56. The meaning of COX-2 inhibitor in this invention also includes SC299 referred to as a fluorescent diaryloxazole. C. Lanzo et al, “Fluorescence quenching analysis of the association and dissociation of a diarylheterocycle to cyclooxygenasel-1 and cyclooxygenase-2: dynamic basis of cycloxygenase-2 selectivity”, Biochemistry May 23, 2000 vol. 39(20):6228-34, and in J. Talley et al, “4,5-Diaryloxazole inhibitors of cyclooxygenase-2 (COX-2)”, Med. Res. Rev. May 1999; 19(3): 199-208. The meaning of COX-2 inhibitor in this invention also includes valdecoxib, See, “4-[5-Methyl-3-phenylisoxazol-1-yl]benzenesulfonamide, Valdecoxib: A Potent and Selective Inhibitor of COX-2”, J. Med. Chem. 2000, Vol. 43: 775-777, and parecoxib, sodium salt or parecoxib sodium, See, N-[[(5-methyl-3-phenylixoxazol-4yl)-phenyl]sulfonyl]propanimide, Sodium Salt, Parecoxib Sodium: A Potent and Selective Inhibitor of COX-2 for Parenteral Administration”, J. Med. Chem. 2000, Vol. 43: 1661-1663. The meaning of COX-2 inhibitor in this invention also includes the substitution of the sulfonamide moiety as a suitable replacement for the methylsulfonyl moiety. See, J. Carter et al, Synthesis and activity of sulfonamide-substituted 4,5-diaryl thiazoles as selective cyclooxygenase-2 inhibitors”, Bioorg. Med. Chem. Lett Apr. 19, 1999: Vol. 9(8): 1171-74, and compounds referenced in the article “Design and synthesis of sulfonyl-substituted 4,5-diarylthiazoles as selective cyclooxygenase-2 inhibitors”, Bioorg. Med. Chem. Lett Apr. 19, 1999: Vol. 9(8): 1167-70. The meaning of this invention includes a COX-2 inhibitor, NS398 referenced in two articles: Attiga et al, “Inhibitors of Prostaglandin Synthesis Inhibit Human Prostate Tumor Cell Invasiveness and Reduce the Release of Matrix Metalloproteinases”, 60 Cancer Research 4629-4637, Aug. 15, 2000, and in “The cyclooxygenase-2 inhibitor celecoxib induces apoptosis by blocking Akt activation in human prostate cancer cells independently of Bcl-2,” Hsu et al, 275(15) J. Biol. Chem. 11397-11403 (2000). The meaning of COX-2 inhibitor in this invention includes the cyclo-oxygenase-2 selective compounds referenced in Mitchell et al, “Cyclo-oxygenase-2: pharmacology, physiology, biochemistry and relevance to NSAID therapy”, Brit. J. of Pharmacology (1999) vol.128: 1121-1132, see especially p. 1126. The meaning of COX-2 inhibitor in this invention includes so-called NO-NSAIDs or nitric oxide-releasing-NSAIDs referred to in L. Jackson et al, “COX-2 Selective Nonsteriodal Anti-Inflammatory Drugs: Do They Really Offer Any Advantages?”, Drugs, June, 2000 vol. 59(6): 1207-1216 and the articles at footnotes 27, and 28. Also included in the meaning of COX-2 inhibitor in this invention includes any substance that selectively inhibits the COX-2 isoenzyme over the COX-1 isoenzyme in a ratio of greater than 10 to 1 and preferably in ratio of at least 40 to 1 as referenced in Winokur WO 99/20110, and has one substituent having both atoms with free electrons under traditional valence-shell-electron-pair-repulsion theory located on a cyclic ring (as in the sulfylamine portion of celecoxib), and a second substituent located on a different ring sufficiently far from said first substituent to have no significant electron interaction with the first substituent. The second substituent should have an electronegativity within such substituent greater than 0.5, or the second substituent should be an atom located on the periphery of the compound selected from the group of a halogen F, Cl, Br or I, or A group VI element S or O. Thus for purposes of this last included meaning of a COX-2 inhibitor, one portion of the COX-2 inhibitor should be hydrophilic and the other portion lipophilic. Also included as a COX-2 inhibitor are compounds listed at page 553 in Pharmacotherapy, 4^{th} ed: A Pathophysiologic Approach, Depiro et al (McGraw Hill 1999) including nabumetone and entodolac. Recognizing that there is overlap among the selective COX-2 inhibitors set out in this paragraph, the intent of the term COX-2 inhibitor is to comprehensively include all selective COX-2 inhibitors, selective in the sense of inhibiting COX-2 over COX-1. The package inserts for rofecoxib and celecoxib are attached and adopted herein by reference. The inventors add to the class of COX-2 inhibitors useful in the invention the drug bearing the name etoricoxib referenced in the Wall Street Journal, Dec. 13, 2000 manufactured by Merck. See, also, Chauret et al, “In vitro metabolism considerations, including activity testing of metabolites, in the discovery and selection of the COX-2 inhibitor etoricoxib (MK-0663),” Bioorg. Med. Chem. Lett. 11(8): 1059-62 (Apr. 23, 2001). Another selective COX-2 inhibitor is DFU [5,5-dimethyl-3-(3-fluorophenyl)-4-(4-methylsulphonyl)phenyl-2(5H)-furanone] referenced in Yergey et al, Drug Metab. Dispos. 29(5):638-44 (May 2001). The inventors also include as a selective COX-2 inhibitor flavanolignanes (sometimes also called flavonoids) which have selective COX-2 inhibitory activity over COX-1 inhibitory activity, including the flavanoid antioxidant silymarin itself, and an active ingredient in silymarin, silybinin, which demonstrated significant COX-2 inhibition relative to COX-1 inhibition. The silymarin also showed protection against depletion of glutathione peroxidase. Zhao et al, “Significant Inhibition by the Flavonoid Antioxidant Silymarin against 12-O-tetracecanoylphorbol 13-acetate-caused modulation of antioxidant and inflammatory enzymes, and cyclooxygenase 2 and interleukin-1 alpha expression in SENCAR mouse epidermis: implications in the prevention of stage I tumor promotion,” Mol. Carcinog. December 1999, Vol 26(4):321-33 PMID 10569809. Silymarin has been used to treat liver diseases in Europe. Bombardelli et al, U.S. Pat. No. 5,912,265, Jun. 15, 1999, and Bombardelli et al, U.S. Pat. No. 6,218,369, Apr. 17, 2001 list compounds having similar characteristics and related to silymarin intended to be included as COX-2 inhibitors in this invention, including silymarin, silibinin, silidianin, silicristin, dehydrosilybin, and phospholipid complexes of one of those flavolignanes. The minimum recommended dose in the therapeutic window is 200-250 mg/day of those compounds.

The term COX-2 inhibitor includes all pharmaceutically acceptable salts for the selective COX-2 inhibiting compound selected. Examples of such salt forms of COX-2 inhibitors include but are not limited to salts derived from inorganic bases including aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamide, 2-diethylaminoethanol, 2-dimethylaminoethanol, ethanolamine, ethylenediamine, N-ethylmorpholine, N-ethylpiperidine, glutamine, glucosamine, histidine, hydrabamine, isopropylamine, lysine, methyglucamine, morpholine, piperazine, piperidine, polyamine resins, procaine, purine, theobromine, triethylamine, trimethylamine, tripropylamine, troethamine, and the like.

The inventors also note the need for and claim a composition potentially including Selenium, and the method of administration potentially including Selenium, if a therapeutic window of Selenium in a patient is not present. See, Brooks and Nelson, Cancer Prevention and Control, Chemoprevention of Cancer at 369 (Marcel Dekker 1995). Selenium can be toxic, but there does need to be an adequate level of Selenium. The patient should be monitored and Selenium supplement given to achieve a therapeutic window for Selenium level to achieve the desired effect of allowing normal functioning of the glutathione pathway and maintaining integrity. In a normal healthy male, the adequate level is approximately 70 micrograms/70 kg of weight. The preferred mode would be a supplement in sequence with cystine administration, but a dose of any part of the invention could include Selenium. The method of treatment could include a sequential or simultaneous dose with either the cystine or the COX-2 inhibitor or both. However, toxic levels of selenium must be avoided. Thus, adequate level means only adequate level.

Magnesium sulfate or Magnesium chloride can be obtained from Spectrum Chemical of Gardena, Calif., and must be at U.S. Pharmacoepia standards.

### BACKGROUND

One of the most important, but underestimated and understudied biochemical cycles in the body is the glutathione cycle. Recently, literature is beginning to recognize the importance of this cycle as the central cycle in determining a body's ability to fight disease. A substance called L-cystine, generally called cystine, and other intermediates in the glutathione cycle called cysteine, and substances convertible into cystine and cysteine such as N-acyl-cysteine, are proposed as part of this invention to enhance immune system competency.

In total, this invention proposes to use a novel combination to inhibit key biochemical cycles in a way that causes a decrease in long-term inflammation, by reducing the mediators of chronic inflammation in both the leukotriene pathway and the White Blood Cell immune response pathway. In that white blood cell immune pathway, a series of cytokines are favorably medicated most notably with respect to TH2, a chronic inflammatory pathway. The net effect results in a more efficient immune response allowing the body to respond to immunologic stimuli with a less damaging and more productive response. Applications include those listed in Arison, U.S. Pat. No. 5,952,347, as well as improved responses in patients with cancer, by way of up regulating decreased TH1 responses.

A second object is to further selectively modify a biochemical cycle that targets inflammatory mechanisms in the body. One of the most damaging aspects of cancer cells is that they trigger an extended inflammatory response in the body. Prostaglandins are some of the most important signals to cause inflammatory responses. The biochemical cycle that we propose to selectively inhibit is an important cycle that converts arachidonic acid to several forms of prostaglandins. That cycle is the cyclooxygenase or COX cycle and proceeds to involve products of 5-LOX (lipooxygenase) pathway called leukotrienes.

Biochemical cycles have many intermediate steps in them and the intermediate compounds are known as “intermediates.” One of those intermediates in the cyclooxygenase cycle is prostaglandin H2 synthase, which has two forms: COX-1 and COX-2. COX-1 is known as a “housekeeping substance” which helps generate substances that protect the stomach. Ding et al, “Blockade of Cyclooxygenase-2 Inhibits Proliferation and Induces Apoptosis in Human Pancreatic Cancer Cells, vol. 20 AntiCancer Research, 2625-2632 (2000). Aspirin inhibits COX-1 and therefore, because it inhibits a substance that protects the stomach, often has abdominal side effects. Recently, substances have become available that selectively inhibit COX-2 enzymes over COX-1 enzymes. COX-2 enzymes regulate pain, inflammation and fever, i.e. inflammatory mechanisms.

For patients who have cancer (or other diseases featuring cells with disproportionately anaerobic function), cancer cells function with a much higher ratio of anaerobic to aerobic function than do normal cells, almost to the point of “anaerobic function”. Because normal body cells have a relatively aerobic function highly dependent on glutathione, the enhancement of the glutathione pathway has a disproportionately beneficial effect on normal cells than enhancement of the glutathione pathway in a cancer cell.

With respect to cystine, the inventors additionally observe that the effect of the COX-2 inhibitor will be enhanced because the enhancement of the glutathione level and pathway has a second important and unexpected effect. The avoidance of a glutathione deficiency steers the patient to have a higher Th-1 response to Th-2 response ratio than the patient would have with any glutathione deficiency. Peterson, J. et al, “Glutathione levels in antigen-presenting cells modulate Th1 versus Th2 response patterns,” Vol 95(6), Proceedings Nat'l Acad. Sci. USA p. 3071-76 (Mar. 17, 1998). This enhancement is independent of, but corollary to the COX-2 inhibitor.

The anti-inflammatory effects and anti-oxidative effects of cystine which correlate with its enhancement of the immune system also moderate any adverse side effects from a selective COX-2 inhibitor and are claimed as a composition and method and method of manufacturing with the selective COX-2 inhibitor. Such adverse side effects that will be moderated relate to negative gastrointestinal, liver or kidney effects.

### DOSAGE

The oral dosage for the preferred combination of leukotriene receptor antagonist utilizes SINGULAIR and cystine.

The amount of SINGULAIR to utilize is evident from the FDA-approved package insert for SINGULAIR.

The amount of cystine to be included in an oral dosage combination is a therapeutically effective amount to reach normal glutathione levels. Such therapeutically effective amount should be initially be 140 mg/70 Kg man twice per day.

More optimally and in an alternate preferred mode, the enhancement by cystine of preferred inflammatory response should enable the reduction of the use of the dose of SINGULAIR. The suggested reduction is an even fractional amount in the therapeutic window of the combination. That is, if the recommended dose of SINGULAIR is 50 mg, the first recommended adjustment is to 40 mg. Similarly, in another preferred mode, with a selective COX-2 inhibitor, the recommended combination is one half the recommended dose of the selective COX-2 inhibitor, and reduction of the recommended dose of SINGULAIR as before from the package insert recommendation. The patient is most desirably weaned to the lowest levels of SINGULAIR in favor of cystine, or in favor of cystine and a selective COX-2 inhibitor. More generally, cystine and a selective COX-2 inhibitor from a naturally occurring compound, such as silimarin or silibinin, are preferably anti-inflammatories to prescription drugs, and the patient is best served by using the combination with the minimum amount of artificial prescription drug.

In other alternative combinations with prescription drugs discussed momentarily, the montelukast (sodium) or other leukotriene antagonist and other prescription drug should be reduced to the lowest dosage in the therapeutic window to achieve efficacy in favor of higher doses of cystine, and the minimum recommended dose of a selective COX-2 inhibitor, in that order.

The cystine can be adjusted by measuring glutathione levels, and if deficient, by increasing the dose in a therapeutic amount.

The use of the cystine in a form such as NAC, in combination with the leukotriene inhibitor in an intravenous dose will have an additional benefit in severely ill cases such as septic shock. NAC alone has been demonstrated to benefit patients with lung deficiency due to septic shock and requiring ventilator support (Chest, 113(6):1616-24 June 1998). According to that article, in that report from a major hospital in Canada, NAC alone reduced the ventilator time for people with septic shock from 21 days mechanical breathing to about 7 days. The intravenous dosage of NAC is 150 mg/Kg in 250 ml of D5W over 15 minutes followed by continuous infusion of 50 mg/kg in 500 ml over 4 hr. This may be combined with the leukotriene inhibitor dosage of 0.001 mg to about 25 mg (preferably from 0.01 mg to about 1 mg) per kg of body weight per day. The dosage will be useful in the treatment of chronic obstructive pulmonary disease which also as glutathione has been demonstrated to be an important factor in the pathophysiology of chronic obstructive pulmonary disease (Chest May 2000; 117(5 Suppl 1):303S-17S). Oxidants/antioxidants and COPD. MacNee W. Edinburgh Lung Environmental Group Initiative, Colt Research Laboratories, University of Edinburgh, Edinburgh, Scotland, UK.

To enhance the effect of the combination already described, the addition of magnesium sulfate for intravenous use is described. For severe situation, Magnesium sulfate IV for 70 kg man is three grams in 30 minutes with steady monitoring of blood pressure and in children may be given at 40/mg/kg over 20 minutes with steady monitoring of blood pressure (Arch Pediatr Adolesc Med October 2000; 154(10):979-83). A 70 kg person should have less than 2 grams per 24 hours. For emergency situations, cystine administered intravenously has a powerful anti-inflammatory effect and could be accompanied by a lower dose of SINGULAIR.

Notably, the combination shows merit with magnesium sulfate for migraine relief. Demirkaya S, et al Headache February 2001; 41(2):171-177. Efficacy of Intravenous Magnesium Sulfate in the Treatment of Acute Migraine Attacks. 1 gram was proposed for what were apparently adults to be administered intravenously over 15 minutes which dose is proposed for this combination if administered intravenously.

For angina, efficacy of magnesium has been shown. The anti-inflammatory and leukotriene inhibition effects of the invention will be useful in conjunction with magnesium. Teragawa H, Kato M, Yamagata T, Matsuura H, Kajiyama G., First Department of Internal Medicine, Hiroshima University School of Medicine, Hiroshima, Japan, The preventive effect of magnesium on coronary spasm in patients with vasospastic angina, Chest December 2000; 118(6):1690-5. For use where a composition for intravenous administration is employed, a suitable dosage range for anti-asthmatic, anti-inflammatory, or anti-allergic use is from about 0.001 mg to about 25 mg (preferably from 0.01 mg to about 1 mg) of SINGULAIR per kg of body weight per day and for cytoprotective use from about 0.1 mg to about 100 mg (preferably from about 1 mg to about 100 mg and more preferably from about 1 mg to about 10 mg) of a compound of SINGULAIR per kg of body weight per day. Aerosol dosing of the combination of the leukotriene inhibitor plus cystine in a form such as NAC is also useful. However, for tachycardia, no showing has been made of effectiveness of magnesium alone. This invention should prove efficacious. In a study reported in Am J Cardiol Dec. 1, 2000; 86(11): 1270-2, A9 entitled “Intravenous magnesium sulfate for acute termination of sustained monomorphic ventricular tachycardia associated with coronary artery disease,” Farouque H M, Sanders P, Young G D, “the efficacy of intravenous magnesium in terminating sustained monomorphic ventricular tachycardia was examined in this study. This therapy was found to be ineffective in aborting monomorphic ventricular tachycardia induced in the electrophysiology laboratory.”

The prior discussion reviews the preferred mode of the invention, a leukotriene receptor antagonist and cystine. Lipoic acid can be an adjunct to the cystine. Cystine is being used to enhance the immune system competency and assist normal cells, through the glutathione pathway, in maintaining their stability.

Because the SINGULAIR is a leukotriene inhibitor, inhibits the action of leukB-4, which in turn stimulates the production of IL-6 (Blood Aug. 15, 1992; 80(4):1004-11). IL-6 is a significant prognostic indicator of survival of prostate cancer (Clin Cancer Res July 2000; 6(7):2702-6).

The instant pharmaceutical combination comprising a leukotriene receptor antagonist inhibitor and cystine includes administration of a single pharmaceutical dosage formulation which contains both the leukotriene receptor antagonist inhibitor and cystine, as well as administration of each active agent in its own separate pharmaceutical dosage formulation. A cystine supplement taken at a different time of day may be a separate dose without the leukotriene receptor antagonist inhibitor. Where separate dosage formulations are used, the leukotriene receptor antagonist inhibitor can be administered at essentially the same time, i.e., concurrently, or at separately staggered time, i.e., sequentially. Where separate dosage formulations are used, the leukotriene receptor antagonist and cystine can be administered at essentially the same time, i.e., concurrently, or at separately staggered time, i.e., sequentially. The instant pharmaceutical combinations are understood to include all these regimens. Administration in these various ways is suitable for the present invention as long as the beneficial pharmaceutical effect of the leukotriene receptor antagonist and cystine are realized by the patient at substantially the same time. If administered without cystine, administration in these various ways are suitable for the present invention as long as the beneficial pharmaceutical effect of the ingredients to the selected combination are realized by the patient at substantially the same time. Such beneficial effect is preferably achieved when the target blood level concentrations of each active drug are maintained at substantially the same time. As much as possible, a single oral dosage formulation is preferred. A single dosage formulation will provide convenience for the patient, which is an important consideration especially for patients who may be in need of multiple medications. Administration of the leukotriene receptor antagonist inhibitor or cystine can be by tablet, liquid suspension, intravenously or many other pharmaceutically acceptable carriers known by or used by reasonably skilled practitioners in the art of pharmacology or pharmacological manufacturing including by the combinations and methods in the cited Winokur art, PCT Appl. US98/21901, filed Oct. 16, 1998, published as WO99/20110 entitled “Combination Therapy for Reducing the Risks Associated with Cardio and Cerebrovascular Disease” and in Nichtberger, U.S. Pat. No. 6,136,804, Oct. 24, 2000. Other methods of administration are set forth in Arison, U.S. Pat. No. 5,952,347, Sep. 14, 1999. As indicated in Arison, U.S. Pat. No. 5,974,347, this antagonism of the actions of leukotrienes indicates that the compounds and pharmaceutical compositions thereof are useful to treat, prevent, or ameliorate in mammals and especially in humans: 1) pulmonary disorders including diseases such as asthma, chronic bronchitis, and related obstructive airway diseases, 2) allergies and allergic reactions such as allergic rhinitis, contact dermatitis, allergic conjunctivitis, and the like, 3) inflammation such as arthritis or inflammatory bowel disease, 4) pain, 5) skin disorders such as atopic eczema, and the like, 6) cardiovascular disorders such as angina, myocardial ischemia, hypertension, platelet aggregation, and the like, 7) renal insufficiency arising from ischaemia induced by immunological or chemical (cyclosporin) etiology, 8) migraine or cluster headache, 9) ocular conditions such as uveitis, 10) hepatitis resulting from chemical, immunological or infectious stimuli, 11) trauma or shock states such as burn injuries, endotoxemia, and the like, 12) allograft rejection, 13) prevention of side effects associated with therapeutic administration of cytokines such as Interleukin II and tumor necrosis factor, 14) chronic lung diseases such as cystic fibrosis, bronchitis and other small- and large-airway diseases, and 15) cholecystitis.

Thus, the compounds of the present invention may also be used to treat or prevent mammalian (especially, human) disease states such as erosive gastritis; erosive esophagitis; diarrhea; cerebral spasm; premature labor; spontaneous abortion; dysmenorrhea; ischemia; noxious agent-induced damage or necrosis of hepatic, pancreatic, renal, or myocardial tissue; liver parenchymal damage caused by hepatotoxic agents such as CCl.sub.4 and D-galactosamine; ischemic renal failure; disease-induced hepatic damage; bile salt induced pancreatic or gastric damage; trauma- or stress-induced cell damage; and glycerol-induced renal failure. The compounds also exhibit cytoprotective action.

The cytoprotective activity of a compound may be observed in both animals and man by noting the increased resistance of the gastrointestinal mucosa to the noxious effects of strong irritants, for example, the ulcerogenic effects of aspirin or indomethacin. In addition to lessening the effect of non-steroidal anti-inflammatory drugs on the gastrointestinal tract, animal studies show that cytoprotective compounds will prevent gastric lesions induced by oral administration of strong acids, strong bases, ethanol, hypertonic saline solutions, and the like.

Two assays can be used to measure cytoprotective ability. These assays are; (A) an ethanol-induced lesion assay and (B) an indomethacin-induced ulcer assay and are described in EP 140,684.

The active drugs can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. The active drugs may also be delivered by the use of monoclonal antibodies as individual carriers to which the compound molecules are coupled. They may also be coupled with soluble polymers as targetable drug carriers. Such polymers can include polyvinylpyrrolidone, pyran copolymer, polyhydroxy-propyl-methacrylamide-phenol, polyhydroxy-ethyl-aspartamide-phenol, or polyethyleneoxide-polylysine substituted with palmitoyl residues. Furthermore, the active drugs may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. All of these are described in Nichtberger, U.S. Pat. No. 6,136,804, Oct. 24, 2000.

Longer term release methods using castor oil as suggested by Williams, U.S. Pat. No. 6,174,540, Jan. 16, 2001 are also contemplated. For emergency situations, cystine administered intravenously has a powerful anti-inflammatory effect and could be accompanied by a lower dose of SINGULAIR.

Pharmaceutical compositions may also contain as the second active ingredient, prostaglandin antagonists such as those disclosed in EP 11,067 (May 28, 1980) or thromboxane antagonists such as those disclosed in U.S. Pat. No. 4,237,160. They may also contain histidine decarboxylase inhibitors such as a-fluoromethyl-histidine, described in U.S. Pat. No. 4,325,961. The leukotriene antagonists may also be advantageously combined with an H.sub.1- or H.sub.2-receptor antagonist, such as for instance acetamazole, aminothiadiazoles disclosed in EP 40,696 (Dec. 2, 1981), benadryl, cimetidine, famotidine, framamine, histadyl, phenergan, ranitidine, terfenadine, loratadine and like compounds, such as those disclosed in U.S. Pat. Nos. 4,283,408; 4,362,736; and 4,394,508. The pharmaceutical compositions may also contain a K.sup.+/H.sup.+ATPase inhibitor such as omeprazole, disclosed in U.S. Pat. No. 4,255,431, and the like. Compounds of SINGULAIR/montelukast sodium may also be usefully combined with mast cell stabilizing agents, such as 1,3-bis(2-carboxychromon-5-yloxy)-2-hydroxypropane and related compounds described in British Patent Specifications 1,144,905 and 1,144,906. Another useful pharmaceutical composition comprises the SINGULAIR/montelukast sodium compounds in combination with serotonin antagonists such as methysergide, the serotonin antagonists described in Nature, 316, 126-131 (1985), and the like. Each of the references referred to in this paragraph is hereby incorporated herein by reference.

Other advantageous pharmaceutical compositions comprise the SINGULAIR/montelukast sodium compounds in combination with anti-cholinergics such as ipratropium bromide, bronchodilators such as the beta agonist salbutamol, metaproterenol, terbutaline, fenoterol and the like, and the anti-asthmatic drugs theophylline, choline theophyllinate and enprofylline, the calcium antagonists nifedipine, diltiazem, nitrendipine, verapamil, nimodipine, felodipine, etc. and the corticosteroids, hydrocortisone, methylprednisolone, betamethasone, dexamethasone, beclomethasone, and the like.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. The term “prophylactically effective amount “is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. The dosage regimen in combination is selected in accordance with a variety of factors including type, species, age, weight, sex and medical condition of the patient; the severity of the condition to be treated; the route of administration; the renal and hepatic function of the patient; and the particular compound or salt or ester thereof employed. Since two different active agents are being used together in a combination therapy, the potency of each of the agents and the interactive effects achieved by combining them together must also be taken into account. A consideration of these factors is well within the purview of the ordinarily skilled clinician for the purpose of determining the therapeutically effective or prophylactically effective amount.

The invention is not meant to be limited to the disclosures, including best mode of invention herein, and contemplates all equivalents to the invention and similar embodiments to the invention for humans and mammals and veterinary science. Equivalents include all pharmacologically active racemic mixtures, diastereomers and enantiomers of the listed compounds and their pharmacologically acceptable salts.

The methods of administration, sequentially or in single dose are part of the invention. The method of manufacturing the dosages in combination in a pharmaceutical carrier are also claimed and part of the invention. Both are apparent from the description above to the reasonably skilled practitioner.

We claim:

1. A combination for combating inflammatory disease effects, particularly asthma, comprising:
a compound selected from the group of leukotriene antagonists, including montelukast (sodium) and cystine in a pharmaceutically acceptable carrier.

2. The combination according to claim 1, further comprising:
a compound selected from the group of prostaglandin antagonists.

3. The combination according to claim 1, further comprising:
a compound selected from the group of thromboxane antagonists.

4. The combination according to claim 1, further comprising:
a compound selected from the group of histidine decarboxylase inhibitors including a-fluoromethyl-histidine.

5. The combination according to claim 1, further comprising:
a compound selected from the group of H.sub.1-receptor and H.sub.2-receptor antagonists, including acetamazole, aminothiadiazole, benadryl, cimetidine, famotidine, framamine, histadyl, phenergan, ranitidine, terfenadine, and loratadine.

6. The combination according to claim 1, further comprising:
a compound selected from the group of K.sup.+/H.sup.+ATPase inhibitors, including omeprazole,.

7. The combination according to claim 1, further comprising:
a compound selected from the group of mast cell stabilizing agents, including 1,3-bis(2-carboxychromon-5-yloxy)-2-hydroxypropane.

8. The combination according to claim 1, further comprising:
a compound selected from the group of serotonin antagonists including methysergide.

9. The combination according to claim 1, further comprising:
a compound selected from the group of anti-cholinergics including ipratropium bromide.

10. The combination according to claim 1, further comprising:
a compound selected from the group of bronchodilators, including beta agonists, including salbutamol, metaproterenol, terbutaline, and fenoterol.

11. The combination according to claim 1, further comprising:
a compound selected from the group of corticosteroids, including hydrocortisone, methylprednisolone, betamethasone, dexamethasone, and beclomethasone.

12. The combination according to claim 1, further comprising:
a compound selected from the group of calcium antagonists, including nifedipine, diltiazem, nitrendipine, verapamil, nimodipine, and felodipine.

13. The combination according to claim 1, further comprising:
a compound selected from the group of anti-asthmatic drugs theophylline, choline theophyllinate and enprofylline.

14. The combination according to claim 1, further comprising:
Magnesium sulfate.

15. The combination according to claim 1, further comprising:
a selective COX-2 inhibitor.

16. The combination according to claim 16, further comprising:
Magnesium sulfate.

17. The combination according to claim 15, further comprising:
a compound selected from the group of prostaglandin antagonists.

18. The combination according to claim 15, further comprising:
a compound selected from the group of thromboxane antagonists.

19. The combination according to claim 15, further comprising:
a compound selected from the group of histidine decarboxylase inhibitors including a-fluoromethyl-histidine.

20. The combination according to claim 15, further comprising:
a compound selected from the group of H.sub.1-receptor and H.sub.2-receptor antagonists, including acetamazole, aminothiadiazole, benadryl, cimetidine, famotidine, framamine, histadyl, phenergan, ranitidine, terfenadine, and loratadine.

21. The combination according to claim 15, further comprising:
a compound selected from the group of K.sup.+/H.sup.+ATPase inhibitors, including omeprazole,.

22. The combination according to claim 15, further comprising:
a compound selected from the group of mast cell stabilizing agents, including 1,3-bis(2-carboxychromon-5-yloxy)-2-hydroxypropane.

23. The combination according to claim 15, further comprising:
a compound selected from the group of serotonin antagonists including methysergide.

24. The combination according to claim 15, further comprising:
a compound selected from the group of anti-cholinergics including ipratropium bromide.

25. The combination according to claim 15, further comprising:
a compound selected from the group of bronchodilators, including beta agonists, including salbutamol, metaproterenol, terbutaline, and fenoterol.

26. The combination according to claim 15, further comprising:
a compound selected from the group of corticosteroids, including hydrocortisone, methylprednisolone, betamethasone, dexamethasone, and beclomethasone.

27. The combination according to claim 15, further comprising:
a compound selected from the group of calcium antagonists, including nifedipine, diltiazem, nitrendipine, verapamil, nimodipine, and felodipine.

28. The combination according to claim 15, further comprising:
a compound selected from the group of anti-asthmatic drugs theophylline, choline theophyllinate and enprofylline.

29. The combination according to claim 1, further comprising:
Lipoic acid.

30. A method of combating inflammatory disease effects, particularly asthma, comprising the following steps:
administering a compound selected from the group of leukotriene antagonists, including montelukast (sodium) in a pharmaceutically acceptable carrier; and
administering cystine in a pharmaceutically acceptable carrier.

31. The method of combating inflammatory disease effects, according to claim 30, further comprising the following step:
Administering selenium.

32. The method of combating inflammatory disease effects, according to claim 31, particularly severe asthma episodes, further comprising the following step:
Administering magnesium sulfate.

33. The method of combating inflammatory disease effects, according to claim 30, particularly severe asthma episodes, further comprising the following step:
Administering magnesium sulfate.

34. The method of combating inflammatory disease effects, according to claim 30, further comprising the following step:
Administering lipoic acid.

35. A method of combating inflammatory disease effects, particularly asthma, comprising the following steps:
administering a compound selected from the group of leukotriene antagonists, including montelukast (sodium); and
administering cystine in a pharmaceutically acceptable carrier; and
administering a selective COX-2 inhibitor.

36. The method of combating inflammatory disease effects, according to claim 35, further comprising the following step:
Administering lipoic acid.

37. The method of combating inflammatory disease effects, according to claim 35, further comprising the following step:
Administering selenium.
